(19) United States
(12) Patent Application Publication
(10) Pub. No.: US 2016/0367434 A1
(43) Pub. Date: Dec. 22, 2016

(54) Electroacupuncture enhanced acupressure treatment apparatus and associated electrode assembly

(71) Applicant: Mimi Gruber, Houston, TX (US)

(72) Inventor: Mimi Gruber, Houston, TX (US)

(21) Appl. No.: 14/999,703

(22) Filed: Jun. 16, 2016

Related U.S. Application Data
(60) Provisional application No. 62/180,756, filed on Jun. 17, 2015.

(51) Int. Cl. A61H 39/00; A61N 1/04; A61H 39/04

(52) U.S. Cl. CPC A61H 39/002; A61H 39/04; A61N 1/048; A61N 1/0492

(57) ABSTRACT

An electroacupuncture enhanced acupressure treatment apparatus and the associated electrode assembly which facilitates treatments by persons other than a skilled acupuncture professional of injuries using electroacupuncture enhanced acupressure techniques.

### BENEFIT OF PRIOR APPLICATION

This application claims the benefit of the filing date of U.S. provisional patent application No. 62/180,756, entitled “A device to give acupuncture treatments and the benefits of acupuncture treatments at home”, filed on Jun. 17, 2015.

### FIELD

Acupuncture points (also called “acupoints”) are locations on the body that are the focus of acupuncture, acupressure, sonopuncture, and/or laser acupuncture treatment. Acupuncture and acupressure are commonly used for pain relief, though they also may be used for a wide range of other conditions.

In Traditional Chinese Medicine, several hundred acupoints are located along what are referred to as “meridians”. There are also additional “extra points” not associated with a particular meridian.

In traditional acupuncture, thin needles are inserted into the body at one or more acupoints. The principles of acupuncture can also be associated with the application of heat, pressure, or laser light to these same acupoints.

Electroacupuncture is a form of acupuncture where a small electric current is passed between pairs of acupuncture needles. It is believed that this practice augments the use of traditional acupuncture.

The principle disadvantages of acupuncture are associated with the use of the thin needles which penetrate the skin. The present invention relates to a variant of electroacupuncture which does not require the use of penetrating needles, but instead provides physical pressure in the vicinity of one or more acupoints while also applying a small electric current between electrodes which are applied to the surface of the skin in the vicinity of one or more acupoints.

The invention also addresses the problems associated with the relatively high prices of acupuncture treatments from trained professionals and the inconvenience of having to go to acupuncture appointments frequently. This invention allows people to safely and effectively give themselves treatments at home or practically anywhere. Many acupressure products are not effective because they only are designed to be used for one or two specific acupoints. While these specific acupoints are believed to help with a particular issue, but sometimes a different issue will not be helped with those specific acupoints.

### BRIEF DESCRIPTION OF THE DRAWINGS

FIG. 1 is a schematic drawing of an embodiment of the invention.

FIG. 2 is a top view of an embodiment of the electrode assembly.

FIG. 3 is a cross-sectional view of an embodiment of the electrode assembly in FIG. 2.

FIG. 4 is a top view of an another embodiment of the electrode assembly.

FIG. 5 is a cross sectional view of an embodiment of the electrode assembly in FIG. 4.

### SUMMARY

This invention includes an electrode assembly which provides the advantages of electroacupuncture enhanced acupressure treatments.

This invention may be used with a large number of different acupoints due to the nature of the novel electrode design. The electrode assembly includes a sticky surface to permit placement of the assembly almost anywhere on the body.

The design of the electrode assembly with the sticky surface permits the electrode assembly to apply physical pressure at the acupoint as well as deliver an electric current to the acupoint.

The design of the electrode assembly also permits accurate spacing of the electrodes from each other, as more than one electrode may be included in each electrode assembly; the electrodes in each assembly may be maintained at a predetermined spacing from each other.

Also, when used by a patient in conjunction with treatments by a trained acupuncture professional, the patient can continue his/her treatments at home when given the proper points to apply the electrodes.

### DETAILED DESCRIPTION

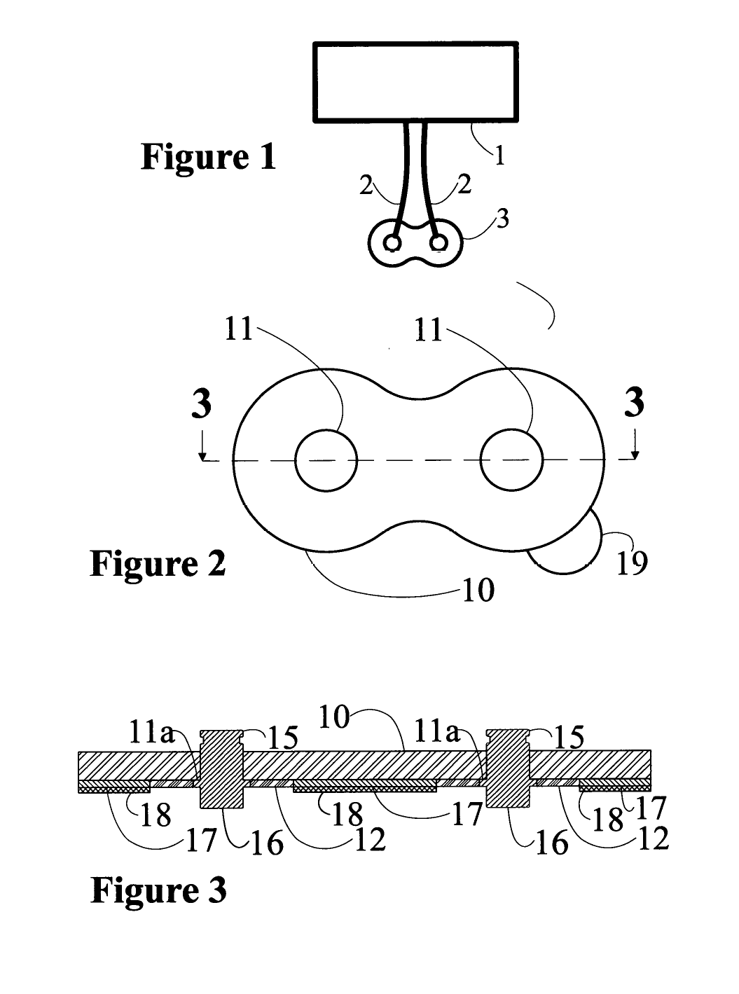

As shown in FIG. 1, the novel electrode assembly is intended to be used in conjunction with a source 1 to provide voltage/current to be delivered through at least one pair of connecting electrical leads 2 to one or more electrode assemblies 3. At least one pair of electrical leads is required to provide the voltage/current to the electrode assembly 3. An electrode assembly 3 is applied at one or more locations on the patient's body corresponding to the desired acupoint(s) to be treated.

As shown in FIGS. 2 and 3, in one embodiment of the invention the electrode assembly 3 would include a body 10 on which is mounted at least two conducting elements 11 are mounted. The body would be made of an electrically insulating material, such as plastic or paperboard. Preferably, the body 10 is sufficiently flexibly to permit application of the electrode assembly to the patient's body while maintaining a predetermined distance between the conducting elements 11.

Each conducting element 11 would extend through the body 10, and have an associated upper portion 15 and lower surface 16. In one embodiment of the invention, one end of the connecting electrical lead 2 may be permanently affixed to (e.g., soldered) to the upper portion 15 of the associated conducting element 11 and the other end capable of being connected to the source 1 (not shown in FIGS. 2 and 3). Preferably, the conducting element 11 will include a shoulder 11a to assist in preventing the body from sliding below the shoulder 11a and facilitate the body applying physical force to the conducting element when the electrode assembly is applied to the patient.

In another embodiment of the invention, one end of the connecting electrical conductor 2 may be attached to the upper portion 15 of the conducting element 11 by any of a variety of releasable electrical connections, including by way of example a pluggable male female electrical connection (such as a jack) or a clip-on, snap-on, screwable, or magnetic connection.

Preferably, each conducting element 11 would be surrounded by a foam pad 12. Each conducting element 11 may be surrounded by a separate foam pad 12, as shown in FIGS. 2 and 3, or in other embodiments a single foam pad may surround more than one conducting element 11. Alternatively, the foam pad may be eliminated if desired.

The electrode assembly 3 also would include a layer of pressure sensitive adhesive 17 on its lower surface to facilitate applying the electrode assembly 3 to the desired location on the patient's body. In most embodiments of the invention, the pressure sensitive adhesive could be covered by a removable protective sheet 18 of material made of a suitable material such as a suitable plastic material or a plastic coated or impregnated paper which could be easily removed to expose the pressure sensitive adhesive prior to application of the electrode assembly 3 to the patient's body. In some embodiments of the invention, an exposed tab 19 on the protective sheet 18 would facilitate removal of the sheet 18 prior to application of the electrode assembly 3 to the patient's body.

The lower surface 16 of the conducting elements 11 would be configured to apply physical pressure body of the patient in the vicinity of the conducting element 11 when applied to the patient. This is accomplished by having the lower surface 16 extend a sufficient distance below the pressure sensitive adhesive to press on the body of the patient and apply physical pressure in the vicinity of the acupoint when the electrode assembly is applied. The lower surface 16 is shaped to be relatively flat (as shown in FIG. 3) or gently curved (as shown in FIG. 5) rather than pointed to avoid penetrating the skin of the patient while applying physical pressure to the acupoint. In other embodiments, the conducting elements 11 may be flared or tapered near the bottom to provide either more or less surface area to apply physical pressure in the vicinity of the acupoint.

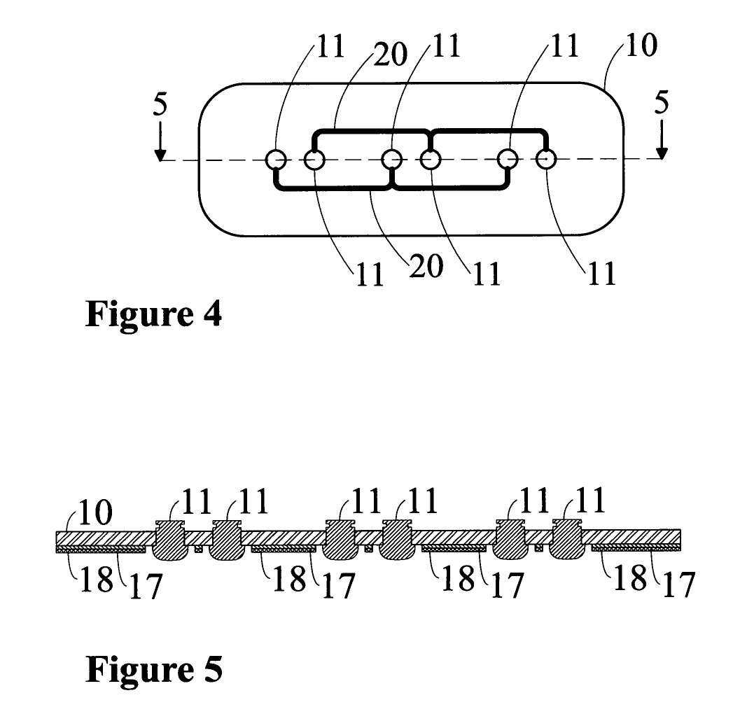

In FIGS. 4 and 5, another embodiment of the invention is shown in which there are more than a single pair of conducting elements 11 mounted on the body 10. Depending on the embodiment, the conducting elements may be wired using bus conductors 20 to function in pairs, as shown in FIG. 4, or may be wired differently, e.g., less than half the conducting elements 11 having one polarity, and the remainder having a neutral or opposite polarity.

What is claimed is:

1. A treatment apparatus comprising:
a source of current/voltage;
at least one electrode assembly comprising
a body made of an electrically insulating material;
the lower side of said body including a layer of pressure sensitive adhesive;
at least one conducting element made of an electrically conducting material, said conducting element penetrating said body and having
an upper portion accessible from above said body and
a lower surface extending below said body, said lower surface extending below said layer of pressure sensitive adhesive; and
at least one electrical conductor forming an electrical connection between said source and said conducting element.

2. The treatment apparatus of claim 1 further comprising at least one foam pad encircling said conducting element.

3. The treatment apparatus of claim 1 having at least two conducting elements which are spaced a predetermined distance from each other.

4. The treatment apparatus of claim 3 further comprising a shoulder located on each said conducting element located below said body.

5. The treatment apparatus of claim 4 in which the electrical connection between said electrical conductor and said conducting element is selected from the group comprised of a pluggable male female, a clip-on, a snap-on, a screwable, or a magnetic connection.

6. An electrode assembly for use in a treatment device comprising:
a body made of an electrically insulating material;
the lower side of said body including a layer of pressure sensitive adhesive;
at least one conducting element made of an electrically conducting material, said conducting element penetrating said body and having
an upper portion adapted to be connected to a conductive lead, and
a lower surface extending below said body, said lower surface extending below said layer of pressure sensitive adhesive.

7. The electrode assembly of claim 6 further comprising at least one foam pad encircling said conducting element.

8. The electrode assembly of claim 6 having at least two conducting elements which are spaced a predetermined distance from each other.

9. The electrode assembly of claim 8 further comprising a shoulder located on each said conducting element located below said body

10. The electrode assembly of claim 9 in which the electrical connection between said electrical conductor and said conducting element is selected from the group comprised of a pluggable male female, a clip-on, a snap-on, a screwable, or a magnetic connection.
